(19) United States
(12) Patent Application Publication
(10) Pub. No.: US 2017/0280987 A1
(43) Pub. Date: Oct. 5, 2017

(54) Intra-oral tongue or cheek retractor with a mirror

(71) Applicant: Molly Weiandt, Anderson, IN (US)

(72) Inventor: Molly Weiandt, Anderson, IN (US)

(21) Appl. No.: 15/419,001

(22) Filed: Jan. 30, 2017

Related U.S. Application Data
(60) Provisional application No. 62/314,476, filed on Mar. 29, 2016.

(51) Int. Cl. A61B 1/247; A61C 5/90; A61B 1/00; A61B 13/00

(52) U.S. Cl. CPC A61B 1/247; A61B 13/00; A61C 5/90; A61B 1/0011; A61C 2201/00

(57) ABSTRACT

Made of a durable and auto-claveable material including a handle portion of the intra-oral tongue/cheek retractor with a mirror device with an offset in the handle, with a handle flat surface on the mirror side, and with a retention end of handle; at least one mirror or mirrored surface; and a manner for securing/holding or having a mirror or mirrored surface to the handle flat surface on the mirror side where the device can free-up hands of a professional and/or an assistant; can remove any extra “hardware” in a mouth of a patient; provides better visual feedback from the mouth of the patient to the professional; is useable for dental procedures and oral surgery; and is sterilizable in conventional dental or surgical machines.

### CROSS-REFERENCE TO RELATED APPLICATIONS

This application claims the benefit of United States Provisional Patent Application with Ser. No. 62/314,476 filed Mar. 29, 2016, by Dr. Molly Weiandt and entitled “Intra-oral tongue or cheek retractor with a mirror”.

### FIELD OF INVENTION

This invention relates generally to dental instrumentation, and in particular, to a cheek, lip and tongue retractor for use during intraoral procedures. The described subject matter relates to oral or dental products in general, and more particularly to intra-oral mirrors. This invention also relates to an intra-oral tongue or cheek retractor with a mirror.

The present invention relates generally to an improved dental mirror that has a new improved configuration having the capability of retracting the tongue and cheek away from the teeth being examined or worked on by the dentist, all with comfort for and safety to the patient. Particularly this device and product is related to oral surgery or dental devices that have a mirrored surfaces and can act as a lip, cheek or tongue retractor or the like. The device is also sterilizable with an auto clave, chemical auto clave, high temperature steamer or similar machines and methods to remove microorganisms or microscopic organisms such as bacteria, viruses, algae and fungi and other germs.

### FEDERALLY SPONSORED RESEARCH

None.

### SEQUENCE LISTING OR PROGRAM

None.

### BACKGROUND

### Problem Solved

### Prior Art

This section is not Applicable to Provisional Applications. However, as far as known, there are no intra-oral tongue or cheek retractor with a mirror or the like. It is believed that this device or product is unique in its design and technologies.

This background as intra-oral tongue or cheek retractor with a mirror should be useful. During intraoral operative procedures, and in particular during dental operative procedures, it is vital to maintain both a clear visual field of the operative site and to prevent iatrogenic damage to the surrounding hard and soft tissue. The proximity of oral soft tissue, such as the tongue, cheek and lip, to the field of work can make it difficult to perform dental procedures. In the past, various dental appliances have been used to isolate or open parts of the mouth to facilitate the performance of dental services. This is most often accomplished by retracting and/or physically preventing the cheek and tongue from contacting or blocking both the operative site and instrumentation such as dental rotary hand pieces (drills), scalers, curettes, scalpels, forceps and the like. Isolation of the cheek, lip and tongue is also required for many non-operative dental procedures, such as the taking of dental impressions.

Dental instruments featuring mirrors or in combination, have long been used in the dental field to assist practitioners with viewing the teeth and gums from the various angles present in the oral cavity. These mirrors have historically been circular in shape and feature specialized handles for maneuverability particularly in the posterior part of the mouth.

### Problem Solved

Many of the concurrent circular mirrored systems have had to suffice for instances when work is performed on a small region of one tooth perhaps for viewing the tooth properly during a procedure. However, these concurrent systems require a Dental assistant to manually hold and place a second mirror or another retracting instrument so that the dental practitioner can then appropriately maneuver the instruments needed for performing the procedure inside the oral cavity.

### PRIOR ART

Examples of prior art in related fields include:
A. U.S. Pat. No. 770,854 entitled “Dental Mouth Prop and Mirror, Cheek Distender” was issued to W. Hare in 1904. It relates to improvements in a dental mouth prop and mirror, cheek-retractor, tongue-protector, and the construction as shown by the old design drawings. It fails to have extendible reach on a handle as in the current invention.
B. U.S. Pat. No. 1,844,733 entitled “Universal Cheek Distender and Tongue Depressor” was issued to J D Wise in 1929. It has for an object the provision of an instrument which combines with a mouth mirror a cheek distender and tongue depressor, so that all surfaces of the teeth of the patient may be plainly reflected. It is a multi-part, complex instrument as compared to the compact design herein provided by Weiandt.
C. U.S. Pat. No. 2,582,121 entitled “Dental Mirror and Retractor” was issued to H. S. Harvey in 1950. It relates to improvements in dental mirrors and retractors and more particularly to the type wherein the mirrors are replaceable and the handles are of conventional structure. Again, this is a multi-part, complex instrument as compared to the compact design herein provided by Weiandt.
D. U.S. Pat. No. 2,653,597 entitled “Tongue Depressor and Mirror Therefor” was issued to W. T. Canan in 1953. It is a described a tongue depressor comprising essentially an elongated substantially flat strip of plastic, with a recess at one end and a translucent mirror having opposite reflecting surfaces located in the recess. It is not offset and has multiple parts with questionable re-use or ability to sterilize like the Weiandt device.
E. U.S. Pat. No. 3,162,191 entitled “Oral Examiner” was issued to W. T. Canan in 1962. Here is shown a tongue depressor with a primary object being the provision of an instrument for use by the medical professions that will, in a single device unite and correlate the functions of a tongue depressor, a retractor, and a laryngeal mirror. It lacks the retractor feature by Weiandt.
F. U.S. Pat. No. 6,939,134 entitled “Cheek, Lip, and Tongue Shield” was issued to Sherry et al. in 2005. The cheek, lip and tongue shield comprising a handle having a first and second end, the first end for gripping; a first shield having an anterior end, a posterior end, and an inferior border, the anterior end transitioning from the handle second end; a non-hinging transverse member having a first and second end, the first end transitioning from the first shield posterior end; and a second shield having an anterior end, a posterior end, and an inferior border, the posterior end transitioning from the transverse member second end. It lacks a mirror or reflecting functionality like Weiandt.
G. U.S. Patent Application US2006/0141415 entitled “Intra-Oral Mirrors” was made by Johnson et al. in 2006. It reveals mirrors for viewing and/or photographing occlusal, buccal, and lingual views of a patient's teeth are described. In an exemplary embodiment, a mirror comprises a first lobe providing an imaging surface during use. It lacks the retraction function shown herein by the applicant.
H. U.S. Patent Application US2008/0160478 entitled “Dental Retractor” was made by Kia in 2008. In accordance with the principles of the present invention, a dental instrument is provided. A pair of arm members are provided, which is in one embodiment a pair of exam mirrors. Each arm member includes a tissue retaining surface which can be the mirror. The arm members are pivotally connected such that, in use, the tissue retaining surfaces contact tissue in a mouth of a patient. It lacks a smooth retraction/depression extension as taught by Weiandt herein.
I. U.S. Patent Application US2011/0256499 entitled “Dental Mirror” was made by Snow in 2011. It shows a dental mirror having an elongate handle and a mirror angularly disposed from one end of said handle. The mirror has an outer perimeter of at least two straight sides—which provides expanded surface area—which meet at a corner having a radius such that the corner retracts the cheek of a dental patient when the mirror is disposed in a viewing position in proximity to the outer side of the gums or teeth. This demonstrates solely a mirror without the offset retractor.
J. U.S. Patent Application US2014/0170590 entitled “Dual Surface Dental Device for Use in Intraoral Photography” was issued to Whalen et al. in 2014. Shown and taught is a tissue retractor, for use by dental practitioners, in order to provide clear viewage of a large sector of the oral cavity. The retractor includes a unibody construction in one embodiment, which is intended for attachment to any of the numerous standard dental instrument handles, with two oblong mirrors connected by a bridge, which is static in some embodiments and retractably dynamic in some embodiments. It is a far larger and more cumbersome device compared with the sleek instrument taught by Weiandt.
K. U.S. Design Patent DES 281.718 entitled “Dental Mirror” was issued to Holstad in 1985. The ornamental design is for a dental mirror, substantially as shown. And shows no offset retractor as in Weiandt.

None of these show nor anticipate the features and functions of the intra-oral tongue or cheek retractor with a mirror.

### SUMMARY OF THE INVENTION

This invention is an intra-oral tongue or cheek retractor with a mirror. Taught here are the ways an intra-oral tongue or cheek retractor may be equipped with a mirror. The combination device may then be used in improved oral surgery and various dental procedures.

The preferred embodiment of an intra-oral tongue or cheek retractor with a mirror is comprised of a durable and auto-claveable material and made of: (a) a handle portion 36 of the intra-oral tongue or cheek retractor with a mirror device 30 with an offset 38 in the handle 36, with a handle flat surface 37A on the mirror 33 side, and with a retention end 39 of handle 36; (b) at least one mirror or mirrored surface 33; and (c) a means 35 for securing/holding or having a mirror or mirrored surface to the handle flat surface 37A on the mirror 33 side.

The newly invented intra-oral tongue or cheek retractor with a mirror may be manufactured at low volumes by very simple means and in high volume production by more complex and controlled systems. The present invention may be fabricated, for example by a casting or molding to create a device of unitary construction, or the handle may be fabricated separately and fastened, for example, by screwing, welding, spot welding or adhering to the remaining components, as described in detail below. The component parts are preferably fabricated from a material that may be sterilized, for example, heat sterilized in, for example, an autoclave or dry heat oven. When surgical stainless steel or other metals are utilized, preferably they and the mirror portion are highly polished to provide a reflective surface.

### Objects and Advantages

There are several objects and advantages of the intra-oral tongue or cheek retractor with a mirror device. There are currently no known intra-oral tongue or cheek retractor with a mirror devices that are effective at providing the objects of this invention.

The intra-oral tongue or cheek retractor with a mirror device has various advantages and benefits are shown in Table A:

| Item | Advantages |
|---|---|
| 1 | Frees-up hands of the professional or assistant |
| 2 | Removes “hardware” in the mouth of the patient |
| 3 | Provides better visual feedback from the mouth of |
| | the patient to the professional |
| 4 | Use for dental procedures or oral surgery |
| 5 | Device is sterilizable in conventional dental or |
| | surgical sterilization apparatuses |

Finally, other advantages and additional features of the present intra-oral tongue or cheek retractor with a mirror device will be more apparent from the accompanying drawings and from the full description of the device. For one skilled in the art of dental procedures and oral surgery instruments, it is readily understood that the features shown in the examples with this product are readily adapted to other types of dental and surgical instruments and devices.

### DESCRIPTION OF THE DRAWINGS—FIGURES

The accompanying drawings, which are incorporated in and constitute a part of this specification, illustrate an intra-oral tongue or cheek retractor with a mirror device that is preferred. The drawings together with the summary description given above and a detailed description given below serve to explain the principles of the intra-oral tongue or cheek retractor with a mirror device. It is understood, however, that the device is not limited to only the precise arrangements and instrumentalities shown.

FIGS. 1 A through 1 D are sketches of the general Intra-oral tongue or cheek retractor with a mirror, components and use.

FIGS. 2 A through 2 D are sketches of the general Intra-oral tongue or cheek retractor with a mirror device with components and features noted.

FIGS. 3 A and 3 B are an additional isometric view and bottom sketch of an Intra-oral tongue or cheek retractor with a mirror device with the components and features shown.

FIGS. 4 A through 4 F are even more sketches of the options to provide the mirror for the Intra-oral tongue or cheek retractor with a mirror device.

FIGS. 5 A through 5 D are sketches of a professional using the Intra-oral tongue or cheek retractor with a mirror device.

FIG. 6 A through 6 D are sketches of prior art tongue and cheek retractors and the like.

FIG. 7 A through 7 C are sketches of other intra oral devices as prior art.

### DESCRIPTION OF THE DRAWINGS—REFERENCE NUMERALS

The following list refers to the drawings:

TABLE B
Reference numbers

| Ref # | Description |
|---|---|
| 30 | intra-oral tongue or cheek retractor with a mirror |
| | device 30 |
| 31 | professional's hand 31 holding an intra-oral tongue or |
| | cheek retractor with a mirror device 30 |
| 33 | mirror or mirrored surface 33 |
| 35 | options and means for securing/holding or having a |
| | mirrored surface 35 |
| 36 | handle portion 36 of Intra-oral tongue or cheek |
| | retractor with a mirror device 30 |
| 37 | handle flat 37 behind/opposite mirror 33 |
| 37A | handle flat surface 37A on the mirror 33 side |
| 38 | offset 38 in handle 36, the angle being various angles |
| | to the handle flat surface 37 (approximately 15, 30, |
| | 45, 60, 75 degrees or the like) |
| 39 | retention end 39 of handle 36 |
| 43 | mouth 43 of patient 44 |
| 44 | patient 44 with Intra-oral tongue or cheek retractor |
| | with a mirror device 30 in mouth 43 of patient |
| | 44 |
| 50 | adhesive 50 |
| 51 | sectioned straps 51 |
| 52 | fully encased channel constraint 52 |
| 53 | polished, reflective surface 53 |
| 55 | adhesive 50 and encased 52 combination 55 |
| 70 | prior art cheek retractor 70 [University of Minnesota |
| | Ear Nose and Throat] |
| 71 | prior art large cheek and tongue retractor 71 |
| | [Hufriedy] |
| 72 | prior art cheek retractor/tissue retractor 72 |
| | [Bishop] |
| 73 | prior art cheek retractor 73 [by Nordent] |
| 74 | prior art 74 U.S. Pat. No. 3,162,191 issued to Canan in |
| | 1962 and entitled “Oral Examiner”. |
| 75 | prior art 75 U.S. Pat. No. 2,653,597 issued to Canan in |
| | 1949 and entitled “Tongue Depressor and Mirror |
| | therfor(e)” |
| 76 | prior art 76 U.S. Pat. No. 2,582,121 issued to Harvey in |
| | 1950 and entitled “Dental Mirror and Retractor” |

### DETAILED DESCRIPTION OF PREFERRED EMBODIMENT

The present development is an intra-oral tongue or cheek retractor with a mirror. This invention relates generally to dental instrumentation, and in particular, to a cheek, lip and tongue retractor for use during intraoral procedures. The described subject matter relates to oral or dental products in general, and more particularly to intra-oral mirrors. This invention also relates to an intra-oral tongue or cheek retractor with a mirror.

The present invention relates generally to an improved dental mirror that has a new improved configuration having the capability of retracting the tongue and cheek away from the teeth being examined or worked on by the dentist, all with comfort for and safety to the patient. Particularly this device and product is related to oral surgery or dental devices that have a mirrored surfaces and can act as a lip, cheek or tongue retractor or the like. The device is also sterilizable with an auto clave, chemical auto clave, high temperature steamer or similar machines and methods to remove microorganisms or microscopic organisms such as bacteria, viruses, algae and fungi and other germs.

The advantages for the intra-oral tongue or cheek retractor 30 with a mirror device 33 are listed above in the introduction. Succinctly the benefits are that the device:
Frees-up hands of the professional or assistant;
Removes “hardware” in the mouth of the patient;
Provides better visual feedback from the mouth of the patient to the professional;
Used for dental procedures or oral surgery;
Device is sterilizable in conventional dental or surgical sterilization apparatuses or machines.

The preferred embodiment of an intra-oral tongue or cheek retractor with a mirror is comprised of a durable and auto-claveable material and made of: (a) a handle portion 36 of the intra-oral tongue or cheek retractor with a mirror device 30 with an offset 38 in the handle 36, with a handle flat surface 37A on the mirror 33 side, and with a retention end 39 of handle 36; (b) at least one mirror or mirrored surface 33; and (c) a means 35 for securing/holding or having a mirror or mirrored surface to the handle flat surface 37A on the mirror 33 side.

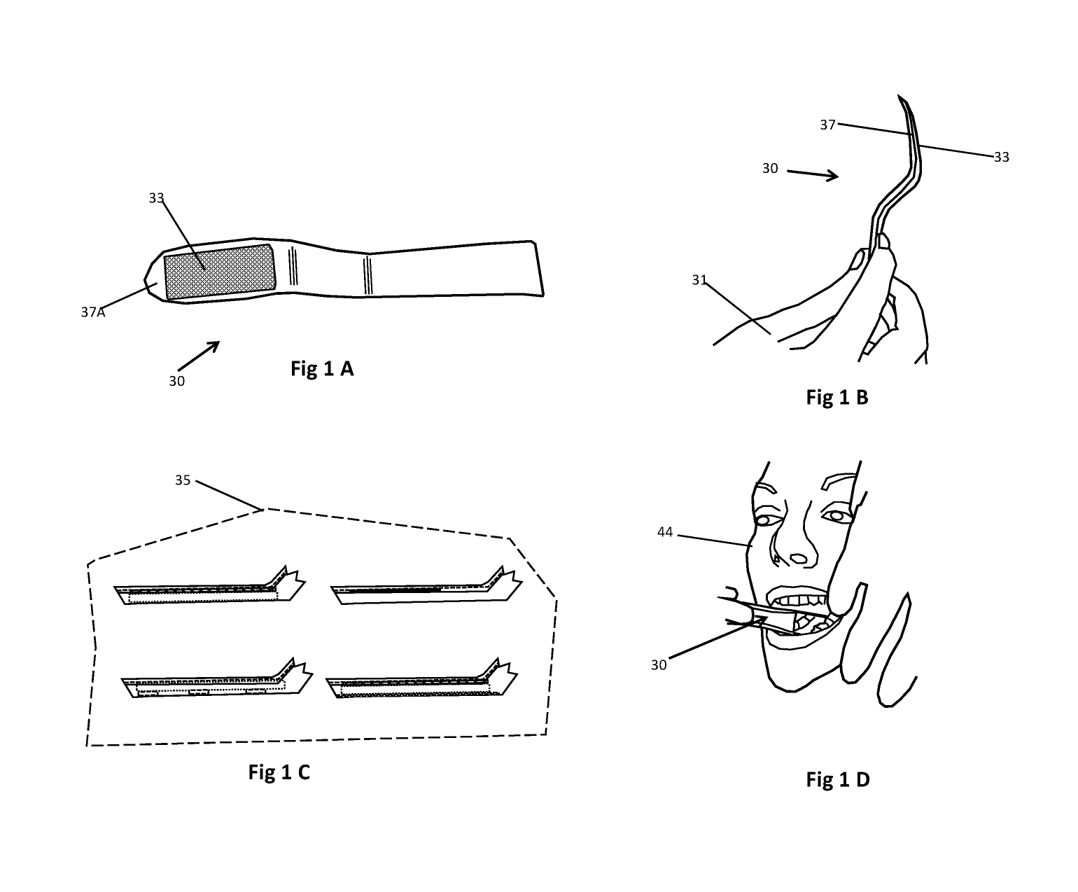

There is shown in FIGS. 1-7 a complete description and operative embodiment of the intra-oral tongue or cheek retractor with a mirror device. In the drawings and illustrations, one notes well that the FIGS. 1-7 demonstrate the general configuration and use of this product. The various example uses are in the operation and use section, below.

The accompanying drawings, which are incorporated in and constitute a part of this specification, illustrate an embodiment of the intra-oral tongue or cheek retractor with a mirror device 30 that is preferred. The drawings together with the summary description given above and a detailed description given below serve to explain the principles of the intra-oral tongue or cheek retractor with a mirror device 30. It is understood, however, that the device 30 is not limited to only the precise arrangements and instrumentalities shown. Other examples of improved dental and surgical devices and uses are still understood by one skilled in the art of such devices to be within the scope and spirit shown here.

FIGS. 1 A through 1 D are sketches of the general Intra-oral tongue or cheek retractor with a mirror, components and use. Demonstrated and shown her are: intra-oral tongue or cheek retractor 30 with a mirror device 33; professional's hand 31 holding an intra-oral tongue or cheek retractor 30 with a mirror device 33; mirror or mirrored surface 33; options and means 35 for securing/holding or having a mirrored surface 33; and patient 44 with intra-oral tongue or cheek retractor 30 with a mirror device 33 in mouth 43 of patient 44.

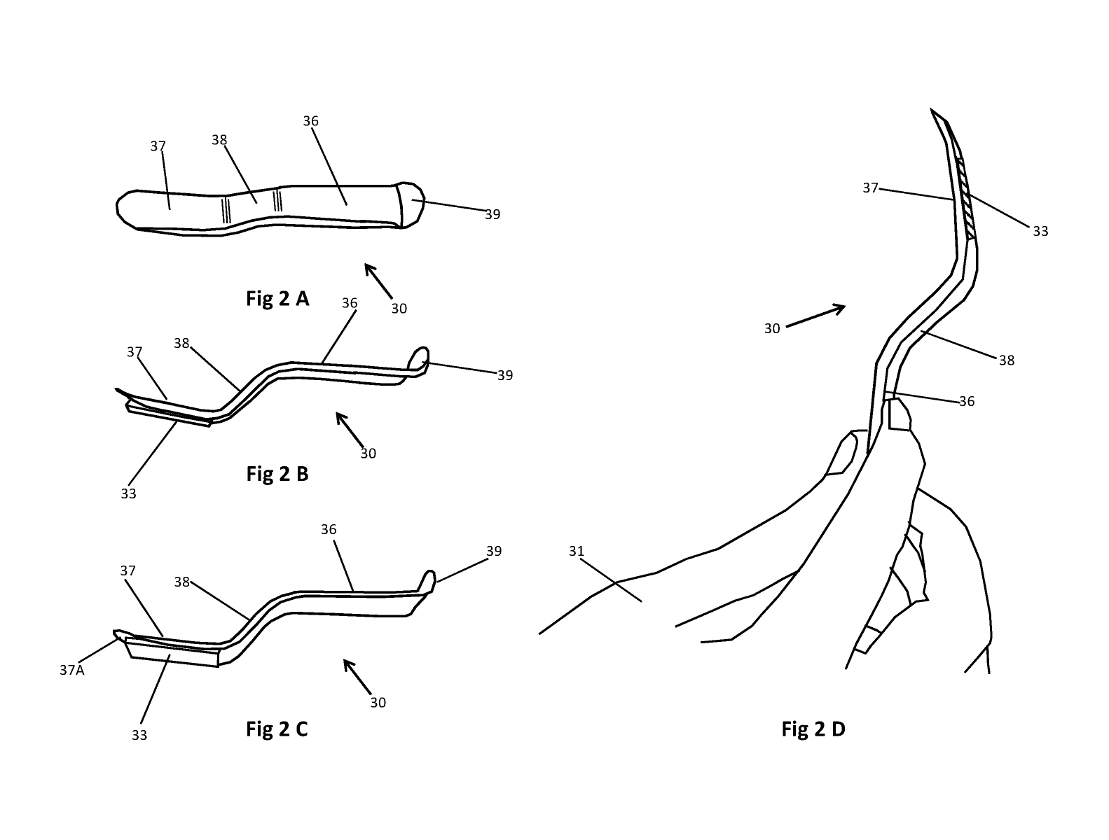

FIGS. 2 A through 2 D are sketches of the general intra-oral tongue or cheek retractor 30 with a mirror device 33 with components and features noted. Provided in these sketches are components and features as follows: intra-oral tongue or cheek retractor 30 with a mirror device 33; professional's hand 31 holding an intra-oral tongue or cheek retractor 30 with a mirror device 33; mirror or mirrored surface 33; handle portion 36 of intra-oral tongue or cheek retractor 30 with a mirror device 33; handle flat 37 behind/opposite mirror 33; handle flat surface 37A on the mirror 33 side; offset 38 in handle 36, the angle of the offset being various angles (approximately 15, 30, 45, 60, 75 degrees or the like) to the handle flat surface 37; and retention end 39 of handle 36. One notes the mirror reflection may be augmented to be a straight reflection—1:1; 2:1 and/or 3:1 reflection as the reflected view to the original. These ratios are as examples and not as a limitation for one skilled in the art of reflective devices.

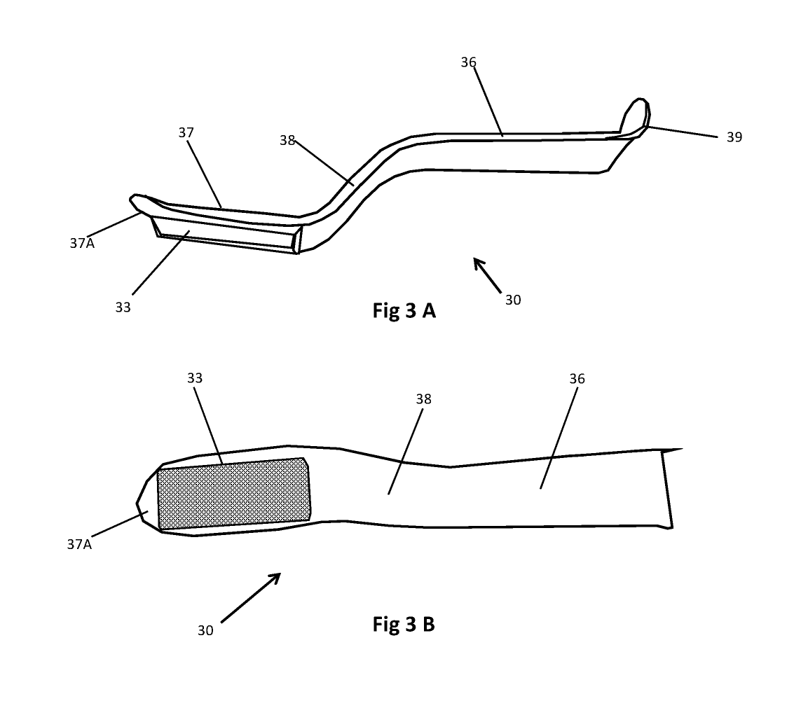

FIGS. 3 A and 3 B are an additional isometric view and bottom sketch of an intra-oral tongue or cheek retractor 30 with a mirror device 33 with the components and features shown. Here are shown: intra-oral tongue or cheek retractor 30 with a mirror device 33; mirror or mirrored surface 33; handle portion 36 of intra-oral tongue or cheek retractor 30 with a mirror device 33; handle flat 37 behind/opposite mirror 33; handle flat surface 37A on the mirror 33 side; offset 38 in handle 36; and retention end 39 of handle 36.

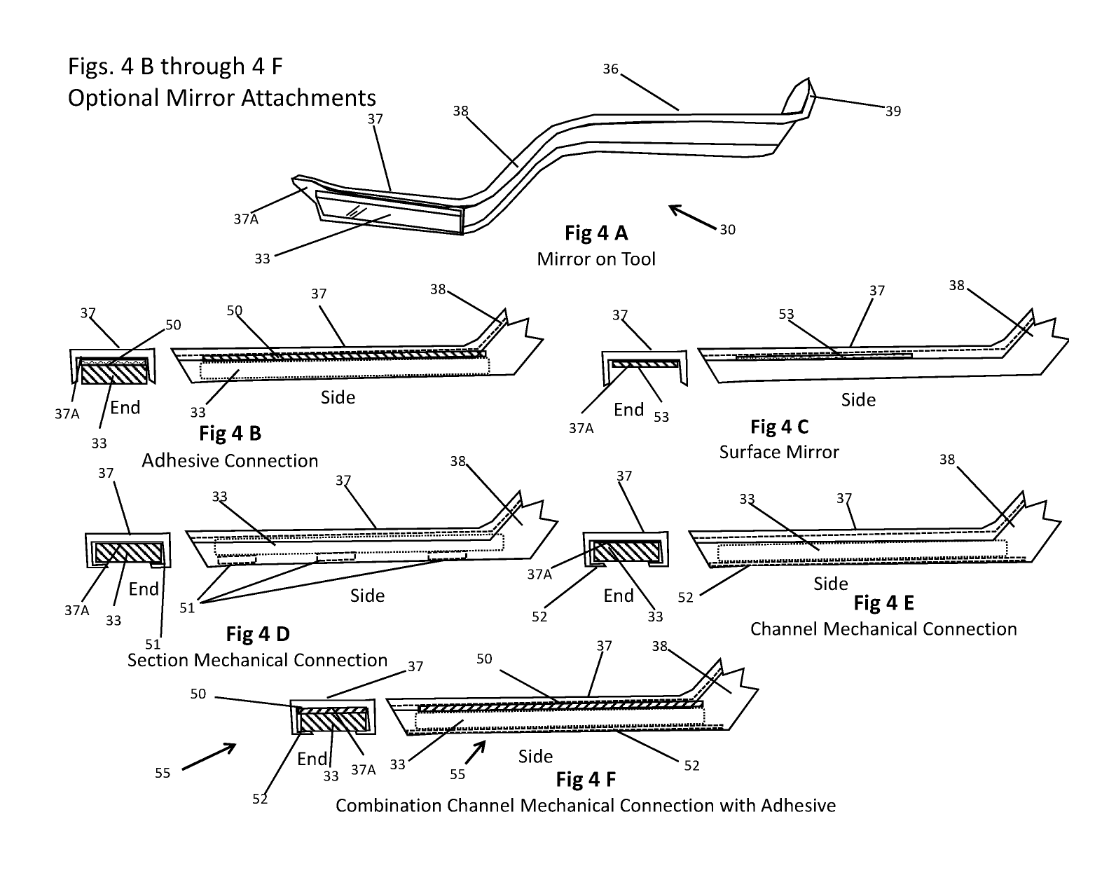

FIGS. 4 A through 4 F are even more sketches of the options to provide the mirror 33 for the intra-oral tongue or cheek retractor 30 with a mirror device 33. In these view are shown: intra-oral tongue or cheek retractor 30 with a mirror device 33; mirror or mirrored surface 33; options and means 35 for securing/holding or having a mirrored surface 33; handle portion 36 of intra-oral tongue or cheek retractor 30 with a mirror device 33; handle flat 37 behind/opposite mirror 33; handle flat surface 37A on the mirror 33 side; offset 38 in handle 36; retention end 39 of handle 36; adhesive 50; sectioned straps 51; fully encased channel constraint 52; polished, reflective surface 53; and an adhesive 50 and encased 52 combination 55.

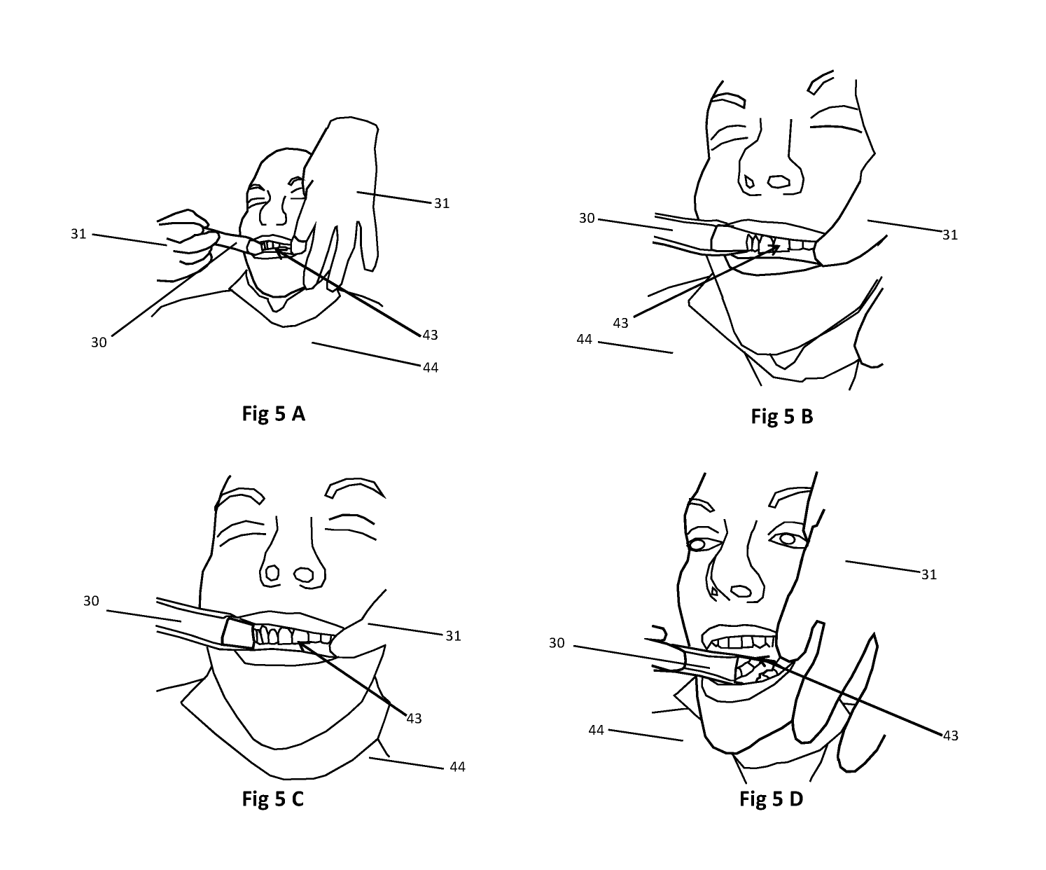

FIGS. 5 A through 5 D are sketches of a professional using the Intra-oral tongue or cheek retractor with a mirror device. These are described below in the operation paragraphs.

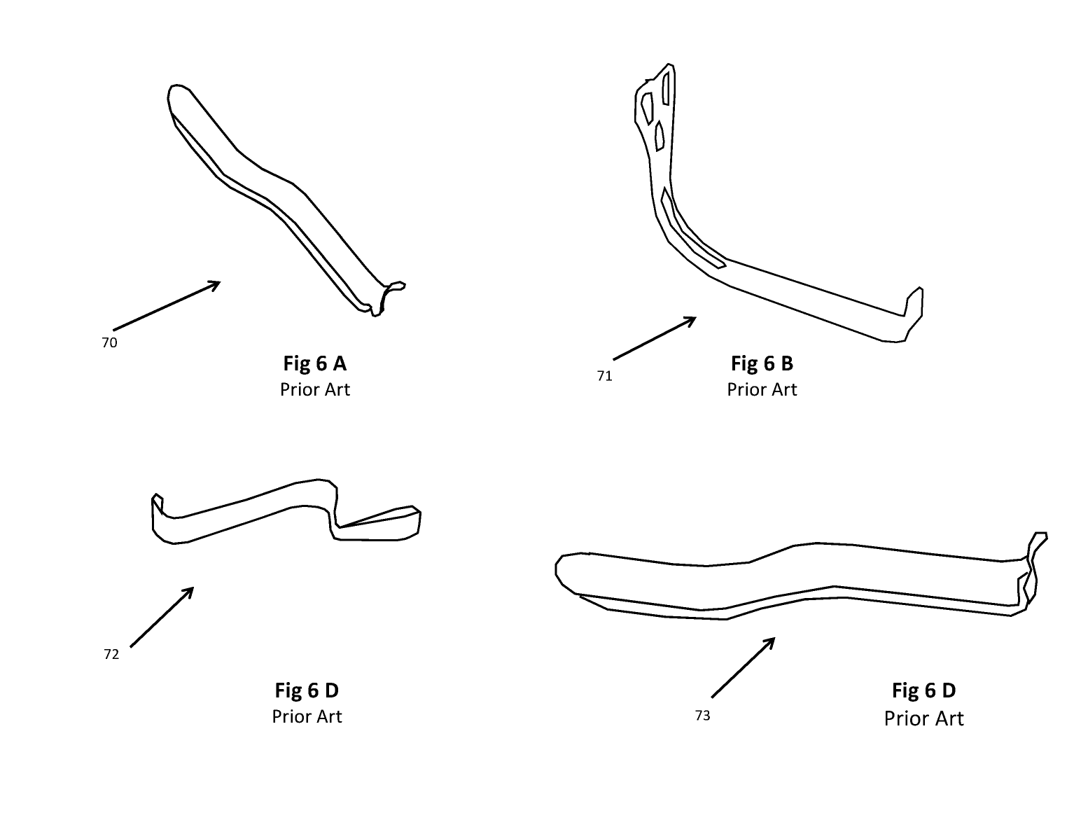

FIG. 6 A through 6 D are sketches of prior art tongue and cheek retractors and the like. Shown here are: prior art cheek retractor 70 [University of Minnesota Ear Nose and Throat]; prior art large cheek and tongue retractor 71 [Hufriedy]; prior art cheek retractor/tissue retractor 72 [Bishop]; and prior art cheek retractor 73 [Nordent].

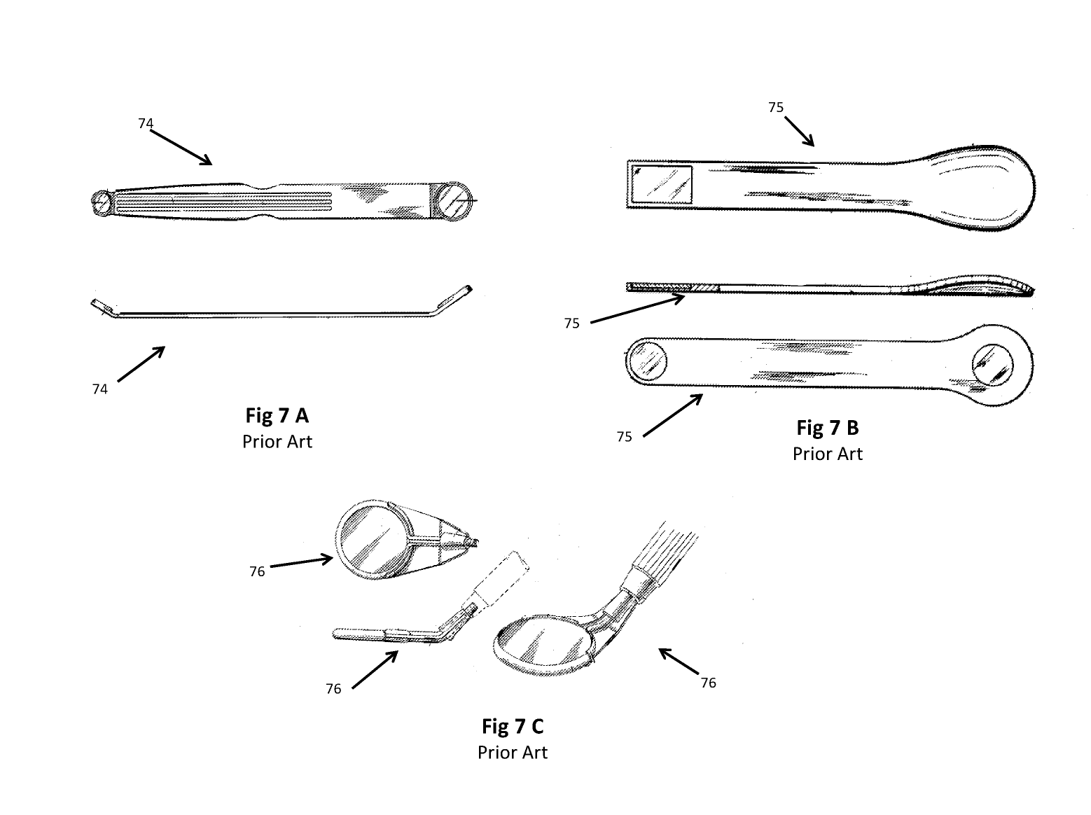

FIG. 7 A through 7 C are sketches of other intra oral devices as prior art. This prior art includes: prior art 74 U.S. Pat. No. 3,162,191 issued to Canan in 1962 and entitled “Oral Examiner”; prior art 75 U.S. Pat. No. 2,653,597 issued to Canan in 1949 and entitled “Tongue Depressor and Mirror therfor(e)”; and prior art 76 U.S. Pat. No. 2,582,121 issued to Harvey in 1950 and entitled “Dental Mirror and Retractor”.

The present invention may be fabricated, for example by a casting or molding to create a device of unitary construction, or the handle may be fabricated separately and fastened, for example, by screwing, welding, spot welding or adhering to the remaining components, as described in detail below. The component parts are preferably fabricated from a non-corrosive, durable material that may be sterilized, for example, heat sterilized in, for example, an autoclave, chemical clawed (chemiclaved) or dry heat oven. When surgical stainless steel or other metals are utilized, preferably they are highly polished to provide a reflective surface. Other anticipated materials are high heat resist and composite and reinforced plastic materials or equal. The materials are also non-allergenic to humans and non-anhydrous once cured (i.e. not attract mouth fluids or blood . . . more of a comfort thought).

The details mentioned here are exemplary and not limiting. Other specific components and manners specific to describing an intra-oral tongue or cheek retractor 30 with a mirror 33 may be added as a person having ordinary skill in the field of the art of dental and surgical instruments and devices and their uses well appreciates.

### Operation of the Preferred Embodiment

The intra-oral tongue or cheek retractor 30 with a mirror 33 has been described in the above embodiment. The manner of how the device operates is described below. One notes well that the description above and the operation described here must be taken together to fully illustrate the concept of the intra-oral tongue or cheek retractor 30 with a mirror 33. The preferred embodiment of an intra-oral tongue or cheek retractor with a mirror is comprised of a durable and auto-claveable material and made of: (a) a handle portion 36 of the intra-oral tongue or cheek retractor with a mirror device 30 with an offset 38 in the handle 36, with a handle flat surface 37A on the mirror 33 side, and with a retention end 39 of handle 36; (b) at least one mirror or mirrored surface 33; and (c) a means 35 for securing/holding or having a mirror or mirrored surface to the handle flat surface 37A on the mirror 33 side.

The intra-oral tongue or cheek retractor 30 with a mirror 33 operates somewhat similar to a conventional dental or surgical retractor but without the need of a separate mirror 33. The professional or his/her assistant need not employ and use a separate mirror from the retractor. Therefore, the use of the new intra-oral tongue or cheek retractor 30 with a mirror 33 enables the professional to have a free hand for probes, drills and other instruments without an assistant's presence to hold the mirror. This provides a simpler procedure and less intrusion into the patient's mouth for the service or procedure.

FIGS. 5 A through 5 D are sketches of a professional hand 31 using the intra-oral tongue or cheek retractor 30 with a mirror device 33. Here are shown sketches of the professional and assistant exploring and administering dental procedures to the patient's mouth. Shown are the intra-oral tongue or cheek retractor 30 with a mirror device 33; professional's hand 31 holding an intra-oral tongue or cheek retractor 30 with a mirror device 33; mouth 43 of patient 44; and the patient 44 with Intra-oral tongue or cheek retractor with a mirror device 30 in mouth 43 of patient 44.

Many uses are anticipated for the intra-oral tongue or cheek retractor 30 with a mirror 33. Some examples, and not limitations, are shown in the following Table C:

| ITEM | DESCRIPTION |
|---|---|
| 1 | Tooth removal |
| 2 | Fillings for teeth |
| 3 | Dental hygiene such as cleanings, |
| | whitening and the like |
| 4 | Preventive procedures such as X- |
| | rays and fluoride treatments |
| 5 | Inspection of teeth and gums |
| 6 | Oral surgery including removal of |
| | teeth, repair of teeth and |
| | implants for teeth as well as gum |
| | repair |

While certain novel features of this invention have been shown and described and are pointed out in the annexed claims, it is not intended to be limited to the details above, since it will be understood that various omissions, modifications, substitutions and changes in the forms and details of the device illustrated and in its operation can be made by those skilled in the art without departing in any way from the spirit of the present invention. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

Unless defined otherwise, all technical and scientific terms used herein have the same meaning as commonly understood by one of ordinary skill in the art to which these inventions belong. Although any methods and materials similar or equivalent to those described herein can also be used in the practice or testing of the present inventions, the preferred methods and materials are now described above in the foregoing paragraphs.

Other embodiments of the invention are possible. Although the description above contains much specificity, these should not be construed as limiting the scope of the invention, but as merely providing illustrations of some of the presently preferred embodiments of this invention. It is also contemplated that various combinations or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Thus, it is intended that the scope of at least some of the present inventions herein disclosed should not be limited by the particular disclosed embodiments described above.

The terms recited in the claims should be given their ordinary and customary meaning as determined by reference to relevant entries (e.g., definition of “plane” as a carpenter's tool would not be relevant to the use of the term “plane” when used to refer to an airplane, etc.) in dictionaries (e.g., widely used general reference dictionaries and/or relevant technical dictionaries), commonly understood meanings by those in the art, etc., with the understanding that the broadest meaning imparted by any one or combination of these sources should be given to the claim terms (e.g., two or more relevant dictionary entries should be combined to provide the broadest meaning of the combination of entries, etc.) subject only to the following exceptions: (a) if a term is used herein in a manner more expansive than its ordinary and customary meaning, the term should be given its ordinary and customary meaning plus the additional expansive meaning, or (b) if a term has been explicitly defined to have a different meaning by reciting the term followed by the phrase “as used herein shall mean” or similar language (e.g., “herein this term means,” “as defined herein,” “for the purposes of this disclosure [the term] shall mean,” etc.). References to specific examples, use of “i.e.,” use of the word “invention,” etc., are not meant to invoke exception (b) or otherwise restrict the scope of the recited claim terms. Other than situations where exception (b) applies, nothing contained herein should be considered a disclaimer or disavowal of claim scope. Accordingly, the subject matter recited in the claims is not coextensive with and should not be interpreted to be coextensive with any particular embodiment, feature, or combination of features shown herein. This is true even if only a single embodiment of the particular feature or combination of features is illustrated and described herein. Thus, the appended claims should be read to be given their broadest interpretation in view of the prior art and the ordinary meaning of the claim terms.

Unless otherwise indicated, all numbers or expressions, such as those expressing dimensions, physical characteristics, etc. used in the specification (other than the claims) are understood as modified in all instances by the term “approximately.” At the very least, and not as an attempt to limit the application of the doctrine of equivalents to the claims, each numerical parameter recited in the specification or claims which is modified by the term “approximately” should at least be construed in light of the number of recited significant digits and by applying ordinary rounding techniques.

With this description it is to be understood that the intra-oral tongue or cheek retractor 30 with a mirror 33 is not to be limited to only the disclosed embodiment of product. The features of the intra-oral tongue or cheek retractor 30 with a mirror 33 are intended to cover various modifications and equivalent arrangements included within the spirit and scope of the description.

What is claimed is:

1. An intra-oral tongue/cheek retractor device with a mirror (30) made of a durable and auto-claveable material and comprised of: (a) a handle portion (36) of the intra-oral tongue/cheek retractor device with a mirror (30) further comprising an offset (38) in the handle portion (36), a flat surface (37A) of the handle portion (36) on a mirror (33) side, and a retention end (39) of the handle portion (36); (b) at least one mirror surface (33); and (c) a means (35) for securing/holding or having a mirror or mirrored surface to the handle flat surface (37A) on the mirror (33) side
wherein the intra-oral tongue/cheek retractor device frees-up hands of a professional or an assistant; removes any extra “hardware” in a mouth of a patient; provides better visual feedback from the mouth of the patient to the professional; is useable for dental procedures and oral surgery uses; and is sterilizable in conventional dental or surgical machines.

2. The intra-oral tongue/cheek retractor device according to claim 1 wherein the durable and auto-claveable material types are selected from the group consisting of surgical stainless steel and high heat resistant composite and reinforced plastic.

3. The intra-oral tongue/cheek retractor device according to claim 2 wherein the durable and auto-claveable material types are non-allergenic to humans and non-anhydrous once the materials are cured.

4. The intra-oral tongue/cheek retractor device according to claim 1 wherein the handle portion (36) can be fabricated by a method selected from the group consisting of metal casting to create a device of unitary construction, molding to create a device of unitary construction, assembly of the handle fabricated separately and fastened to the remaining components by a means for securing.

5. The intra-oral tongue/cheek retractor device according to claim 4 wherein the means for securing is selected from the group consisting of screwing a threaded fastener, welding, spot welding and adhesion of the handle to the remaining components.

6. The intra-oral tongue/cheek retractor device according to claim 1 wherein the mirror can be integrally buffed and polished as a reflective surface (53) into the metal and composite surface of the flat surface (37A).

7. The intra-oral tongue/cheek retractor device according to claim 1 wherein the mirror reflection ratio can be selected from the group consisting 1:1, 2:1, or 3:1 as the reflected view to the original.

8. The intra-oral tongue/cheek retractor device according to claim 1 wherein the mirror can be secured to the flat surface (37A) by the means (35) for securing/holding that can be selected from the group consisting of an adhesive (50); a plurality of sectioned straps (51); fully encased channel constraint (52); and an adhesive (50) and encased (52) combination (55).

9. The intra-oral tongue/cheek retractor device according to claim 1 wherein the angle of the offset (38) in the handle (36) is selected from a group consisting of angles being approximately 15, 30, 45, 60, and 75 degrees from the handle flat surface (37).

10. The intra-oral tongue/cheek retractor device according to claim 1 wherein the use is selected from a group consisting of tooth removal; fillings for teeth; dental hygiene of cleanings and whitening; preventive procedures of X-rays and fluoride treatments; inspection of teeth and gums; and oral surgery including removal of teeth, repair of teeth and implants for teeth as well as gum repair.

11. An intra-oral tongue/cheek retractor device with a mirror (30) made of a stainless steel and comprised of: (a) a handle portion (36) of the intra-oral tongue/cheek retractor device with a mirror (30) further comprising an offset (38) in the handle portion (36), a flat surface (37A) of the handle portion (36) on a mirror (33) side, and a retention end (39) of the handle portion (36); (b) at least one mirror surface (33); and (c) an adhesive for securing/holding a mirror to the handle flat surface (37A) on the mirror (33) side
wherein the intra-oral tongue/cheek retractor device frees-up hands of a professional or an assistant; removes any extra “hardware” in a mouth of a patient; provides better visual feedback from the mouth of the patient to the professional; is useable for dental procedures and oral surgery uses; and is sterilizable in conventional dental or surgical machines.

12. The intra-oral tongue/cheek retractor device according to claim 11 wherein the use is selected from a group consisting of tooth removal; fillings for teeth; dental hygiene of cleanings and whitening; preventive procedures of X-rays and fluoride treatments; inspection of teeth and gums; and oral surgery including removal of teeth, repair of teeth and implants for teeth as well as gum repair.

13. An intra-oral tongue/cheek retractor device with a mirror (30) made of a durable and auto-claveable material and comprised of: (a) a handle portion (36) of the intra-oral tongue/cheek retractor device with a mirror (30) further comprising an offset (38) in the handle portion (36), a flat surface (37A) of the handle portion (36) on a mirror (33) side, and a retention end (39) of the handle portion (36) and (b) at least one mirror surface (33) polished and buffed into the flat portion (37A);
wherein the intra-oral tongue/cheek retractor device frees-up hands of a professional or an assistant; removes any extra “hardware” in a mouth of a patient; provides better visual feedback from the mouth of the patient to the professional; is useable for dental procedures and oral surgery uses; and is sterilizable in conventional dental or surgical machines.

14. The intra-oral tongue/cheek retractor device according to claim 13 wherein the use is selected from a group consisting of tooth removal; fillings for teeth; dental hygiene of cleanings and whitening; preventive procedures of X-rays and fluoride treatments; inspection of teeth and gums; and oral surgery including removal of teeth, repair of teeth and implants for teeth as well as gum repair.
